(19) United States
(12) Patent Application Publication
(10) Pub. No.: US 2008/0112531 A1
(43) Pub. Date: May 15, 2008

(54) Method and Assembly for CBCT Type X-Ray Imaging

(76) Inventor: Juuso Siren, Espoo (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/560,102

(22) Filed: Nov. 15, 2006

(51) Int. Cl. A61B 6/03

(52) U.S. Cl. 378/4

(57) ABSTRACT

The invention relates to an assembly for executing a cone-beam X-ray imaging process. The assembly comprises in the path of X-radiation at least two reference markers (26), which comprise materials whose densities are different from each other. In view of performing a cone-beam X-ray imaging process, the assembly comprises an X-ray source (6) for generating X-radiation and transmitting it to an object (2), a collimator (7) collimator before the object for collimating the X-radiation for a conical or pyramidal beam of rays, a detector (8) capable of receiving X-radiation transmitted through the object for providing X-ray image information, scanner means (16), on the basis of whose movements said X-ray imaging is effected from more than one angle of view, and an image processing unit (12) for utilizing the density information of the reference markers in the processing of X-ray image information.

### FIELD OF THE INVENTION

The invention relates to a method and assembly in CBCT type X-ray imaging (Cone Beam Computed Tomography), said method and assembly being particularly useful in providing images from the head and neck area of a patient.

### TECHNICAL BACKGROUND

While conventional CT type X-ray imaging (Computed Tomography) proceeds by irradiating a presently imaged object across its entire cross-sectional area, the irradiation in CBCT type X-ray imaging is performed with a conical or pyramidal beam of rays, whereby a portion of the object excluded from the beam will not be irradiated.

Successful calibration of a CT type X-ray apparatus is highly important for obtaining sufficiently high-quality X-ray images and for providing preconditions for reliable diagnoses. The calibration of a CT type X-ray apparatus is performed prior to its hospital use by making sure with various known quality control methods that the apparatus is capable of providing sufficiently precise Houndsfield units from substances of varying densities. The Houndsfield units are used to establish a model, which is intended for measuring the transmission of X-radiation and which provides a model of radiation transmission values for imaging elements or voxels in substances of varying densities.

FIG. 1 features what is meant by a voxel. A three-dimensional object 2 to be imaged is divided into voxels 4, which provide a basis for working out a matrix useful in computational 3D image processing. An X-ray source 6 supplies radiation to the object 2. Upon passing through a presently imaged object, the X-rays received by a detector 8 have traveled linearly through clusters of voxels. Accordingly, the intensity of received X-radiation provides a basis for determining how much the X-rays are suppressed by whichever cluster of voxels, in other words the density of a substance present in whichever cluster of voxels.

However, it is not possible to execute the calibration of a cone-beam X-ray apparatus (CBCT apparatus) the same way as that of a CT apparatus as some of the cross-sectional area of a presently imaged object is excluded from the beam of rays. If the calibration of a CBCT apparatus is nevertheless performed in a manner similar to that of a CT apparatus, the resulting calibration of the CBCT apparatus will be deficient.

### BRIEF DESCRIPTION OF THE INVENTION

An assembly of the invention makes it possible to delete such factors from X-ray images produced by cone-beam X-ray imaging, which could lead to deficient or false diagnoses.

The invention relates to a method for executing a cone-beam X-ray imaging process, said method comprising, prior to X-ray imaging, providing the path of X-radiation with at least two reference markers comprising materials with densities different from each other. In view of executing a cone-beam X-ray imaging process, an object is supplied with X-radiation, said X-radiation being collimated upstream of the object for a conical or pyramidal beam of rays. The X-radiation that has passed through the object is received for providing X-ray image information. With said method steps, the cone-beam X-ray imaging will become executed from more than one angle of view. The method utilizes the density information of reference markers in the processing of X-ray image information.

The invention relates also to an assembly for executing a cone-beam X-ray imaging process. The assembly comprises at least two reference markers in the path of X-radiation, said markers comprising materials with densities different from each other. In view of executing a cone-beam X-ray imaging process, the assembly comprises
an X-ray source for generating X-radiation and transmitting it to an object,
a collimator before the object for collimating the X-radiation for a conical or pyramidal beam of rays,
a detector for receiving the X-radiation transmitted through the object for providing X-ray image information,
scanner means, on the basis of whose movements said X-ray imaging is effected from more than one angle of view,
and an image processing unit for utilizing the density information of the reference markers in the processing of X-ray image information.

The invention is based on the fact that, in cone-beam X-ray imaging, the processing of X-ray image information based on density information is possible by utilizing the density information of the reference markers having different densities involved in every X-ray imaging process.

It is not possible to calibrate a CBCT apparatus reliably with a calibration method based on Houndsfield units and similar to what is used for calibrating a CT apparatus. An assembly of the invention is capable of providing a condition in which such need to calibrate a CBCT apparatus does not exist.

### LIST OF FIGURES

FIG. 1 shows the division of a presently imaged object into voxels in three-dimensional X-ray imaging.

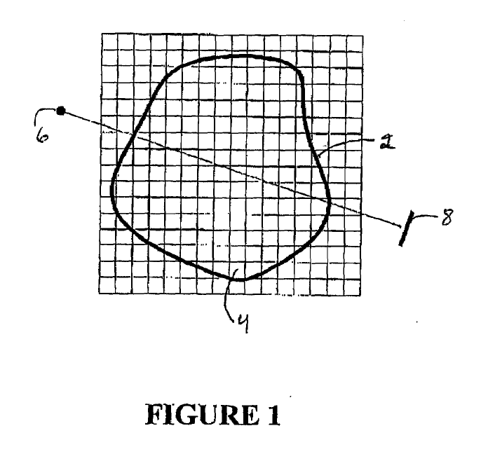

FIG. 2 shows the construction of a CT type cone-beam X-ray apparatus (a CBCT apparatus).

### DETAILED DESCRIPTION OF THE INVENTION

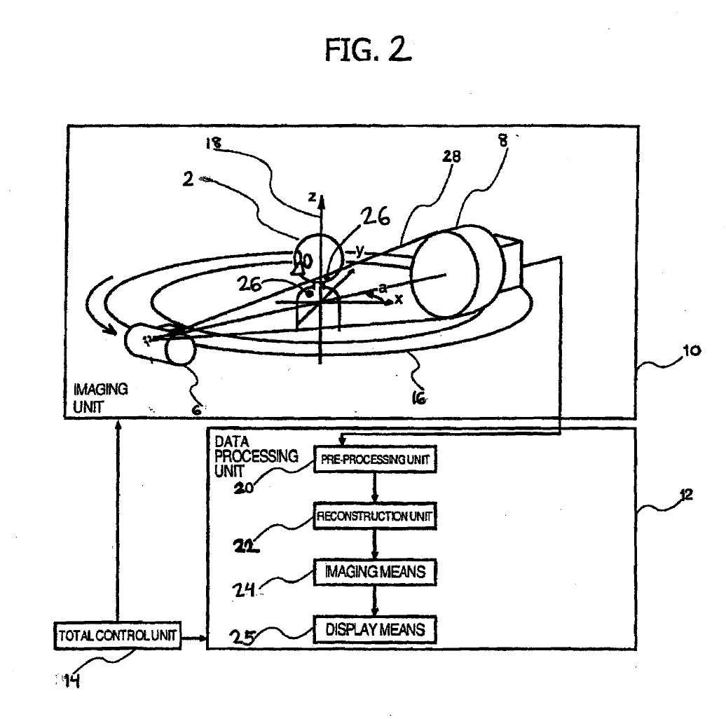

FIG. 2 illustrates the construction of a CT type cone-beam X-ray apparatus. The CT type cone-beam X-ray apparatus is divided into an imaging unit 10 for effecting an imaging procedure and an image processing unit 12 for processing received image data. A control unit 14 performs a total control regarding the imaging unit and the image processing unit. The imaging unit is provided with an X-ray source 6 and a radiation receiving means or detector 8 aligned with each other on the opposite sides of a presently imaged object. Both the X-ray source and the detector are included in a scanner device 16, which circles around an imaged object 2 with an axis of rotation 18 constituting the center of rotation.

In order to capture projection images from a range of viewing angles, the scanner device 16 is rotated to each predetermined angle of view for emitting X-rays from an X-ray source. The scanner device 16 comprises support elements and guide elements for setting the scanner device at various angles of view. The detector 8 performs a measurement for the intensity of emitted X-rays 28 in order to provide X-ray image information. Said X-rays have traveled through the object 2 at said predetermined angles. The received X-ray image information is converted into digital projection image data, for example by means of electronics accompanying the detector, whereafter the image data is sent to the image processing unit 12. The conversion into digital image data can also be executed in the image processing unit.

In the image processing unit, pre-processing means 20 are used for effecting, for example, a gamma correction, a distortion correction, a logarithmic transformation and a non-uniformity correction for the detector 8. The pre-processing means 20 constitute a part of image processing software, yet can also be implemented by way of electronics without actual software structures. The pre-processing sequence is followed by reconstruction means 22 which perform a reconstruction of the three-dimensional image on the basis of assembled pieces of image data. The reconstruction means can be implemented by way of image processing software yet can also be implemented on the basis of electronics without actual software structures. The three-dimensional image can be defined for example by being a three-dimensional X-ray absorption coefficient distribution of an imaged object.

A prior known reconstruction arithmetical operation method useful in a cone-beam CT apparatus is for example Feldkamp's cone-beam reconstruction arithmetical operation method or the like, known from the article L. A. Feldkamp et al.: PRACTICAL CONE-BEAM ALGORITHM, Journal of Optical Society of America, A. Vol. 1, No. 6, pp. 612 to 619 (1984) (article 1).

Finally, the reconstructed three-dimensional image is subjected by imaging means 24 to image processing, such as e.g. volume-rendering or MIP (maximum-intensity-projection) processing for presenting the resulting image as a two-dimensional on a display 25. Thus, image processing is executed by the imaging means on the basis of an angle of view, an area under observation and the like parameters entered through instruction means, such as a keyboard, a mouse, and a tracking ball.

Cone-beam CT type X-ray imaging is carried out in such a way that the scanner device 16, provided with an imaging system involving the X-ray source 6 and the detector 8, is rotated around the object 2. After its reception, the transmitted radiation is imaged and the reconstruction means 22 produce a three-dimensional X-radiation absorption coefficient distribution of the object 2 placed on a stationary coordinate system fixed to a frame of the apparatus. The stationary coordinate system is defined by means of the imaging system, i.e. the z-axis as a center of rotation for the scanner device and the rectangular x- and y-axes on a plane that contains an orbit for the focus of an X-ray source.

The assembly of the invention for performing cone-beam X-ray imaging comprises in the path of X-radiation at least two reference markers 26, which include materials of unequal densities. The reference markers are set in the path of X-radiation for example by attaching the same to the imaged object 2, i.e. to a patient. Regarding the shape thereof, the reference markers can be for example ball-shaped and each reference ball is made of a material whose density is different from others. The reference balls' 26 density information is previously known. In view of executing a cone-beam X-ray imaging process, the assembly comprises the X-ray source 6 for generating X-radiation and transmitting it to an object and the collimator 7 for collimating the X-radiation upstream of the object for a conical or pyramidal beam of rays. The X-radiation transmitted through the object is received by the detector 8 for providing X-ray image information. The scanner device 16 can be adapted to perform the motions for effecting said X-ray imaging from more than one angle of view. In the method of the invention, the previously known density information regarding the reference markers 26, i.e. the reference balls, is utilized in the processing of X-ray image information, which is executed in the image processing unit 12 included in the assembly of the invention.

In first and second preferred embodiments of the invention, the image processing unit 12 comprises means for executing X-ray image information processing steps as follows: X-ray image information is reconstructed followed by LUT (Look-Up-Table) processing for scaling density information to Houndsfield units. The second preferred embodiment of the invention can be implemented also in such a way that the reconstruction of X-ray image information is preceded by adjusting the LUT values of pieces of projection image data assembled from various viewing angles, said values being checked in an iteration process. Both of said embodiments make use of the previously known density values of the reference balls 26, such that certain gray scale values of voxels are given as target values for at least two different density values, which target values are used as a basis for executing an interpolation of the LUT values. Said interpolated LUT values are used as a basis for determining LUT values for other voxel values of the projection image data.

Based on the previously known density information of the reference balls 26, the reconstructed X-ray image information can be processed for determining reconstructed voxel values corresponding to certain absolute densities. LUT values for such reconstructed voxel values are determined by bringing the gray scale values of these particular reconstructed voxel values to match those gray scale values the use of which is deemed most preferred for the relevant absolute density.

Hence, by virtue of the method according to the invention, a precise calibration of Houndsfield values for CBCT imaging is enabled by utilizing X-ray image information reconstructed in the image processing unit 12 and previously known densities of the reference markers 26.

Technical implementations more detailed than the foregoing are not described as those can be executed in terms of hardware engineering, electronics and programming by implementations known from the prior art.

Although the invention has been described above with reference to the accompanying figures and specification, the invention is by no means limited to those as the invention is apt to modifications within the scope allowed by the appended claims.

1. A method for executing a cone-beam X-ray imaging process, characterized in that
an X-ray imaging process is preceded by providing the path of X-radiation with at least two reference markers, comprising materials whose densities are different from each other,
in view of performing a cone-beam X-ray imaging process, X-radiation is collimated for a conical or pyramidal beam of rays and transmitted to an object,
the X-radiation transmitted through the object is received for providing X-ray image information,
by means of said method steps, a cone-beam X-ray imaging process is effected from more than one angle of view,
and the method utilizes the density information of the reference markers in the processing of X-ray image information.

2. A method as set forth in claim 1, characterized in that X-ray image information is reconstructed, followed by LUT (Look-Up-Table) processing for scaling density data to Houndsfield units.

3. A method as set forth in claim 1, characterized in that the reconstruction of X-ray image information is preceded by adjusting LUT values of amounts of X-ray image information assembled from different viewing angles, said values being checked in an iteration process.

4. A method as set forth in claim 1, characterized in that the previously known density values of reference markers are utilized in such a way that certain gray scale values of voxels are given as target values for at least two different density values, which target values are used as a basis for executing an interpolation of the LUT values.

5. A method as set forth in claim 4, characterized in that the interpolated LUT values are used as a basis for determining LUT values for other voxel values of X-ray image information.

6. An assembly for executing a cone-beam X-ray imaging process, characterized in that the assembly comprises in the path of X-radiation at least two reference markers, which comprise materials whose densities are different from each other,
and in view of executing a cone-beam X-ray imaging process, the assembly comprises an X-ray source for generating X-radiation and transmitting it to an object,
a collimator before the object for collimating the X-radiation for a conical or pyramidal beam of rays,
a detector capable of receiving X-radiation transmitted through the object for providing X-ray image information,
scanner means, on the basis of whose movements said X-ray imaging process is effected from more than one angle of view,
an image processing unit for utilizing the density information of the reference markers in the processing of X-ray image information.

7. An assembly as set forth in claim 6, characterized in that the assembly comprises an image processing unit for reconstructing X-ray image information and for performing a post-reconstruction LUT (Look-Up-Table) processing for scaling the density information to Houndsfield units.

8. An assembly as set forth in claim 6, characterized in that the assembly comprises an image processing unit for adjusting the LUT values of amounts of X-ray image information assembled from different viewing angles prior to the reconstruction of X-ray image information and for checking said LUT values in an iteration process.

9. An assembly as set forth in claim 6, characterized in that the assembly comprises an image processing unit for utilizing the previously known density values of the reference markers, such that certain gray scale values of voxels are given as target values for at least two different density values, said target values serving as a basis for executing an interpolation of LUT values.

10. An assembly as set forth in claim 9, characterized in that the assembly comprises an image processing unit for determining LUT values for other voxel values of X-ray image information on the basis of the interpolated LUT values.
